(19) United States
(12) Patent Application Publication
(10) Pub. No.: US 2021/0030267 A1
(43) Pub. Date: Feb. 4, 2021

(54) IMAGE PROCESSING METHOD, PROGRAM, AND IMAGE PROCESSING DEVICE

(71) Applicant: NIKON CORPORATION, Tokyo (JP)

(72) Inventors: Mariko HIROKAWA, Yokohama-shi (JP); Yasushi TANABE, Fujisawa-shi (JP)

(73) Assignee: NIKON CORPORATION, Tokyo (JP)

(21) Appl. No.: 17/072,371

(22) Filed: Oct. 16, 2020

Related U.S. Application Data
(63) Continuation of application No. PCT/JP2019/016656, filed on Apr. 18, 2019.

(30) Foreign Application Priority Data
Apr. 18, 2018 (JP) 2018-080277

(51) Int. Cl. A61B 3/00; G06T 7/00; G06T 7/33; G06T 7/60; G06T 11/00; A61B 3/12

(52) U.S. Cl. CPC A61B 3/0025; G06T 7/0016; G06T 7/337; G06T 7/60; G06T 11/001; A61B 3/0058; G06F 3/04817; G06T 2207/30041; G06T 2207/30104; G06T 2207/20221; G06T 2207/10064; G06T 2200/24; A61B 3/1241

(57) ABSTRACT

An image is created to visualize the efficacy of treatment to a fundus.

An image processing method includes extracting a first frame from a first moving image of an examined eye and extracting a second frame from a second moving image of the examined eye, and comparing the first frame against the second frame.

### CROSS-REFERENCE TO RELATED APPLICATIONS

This application is a continuation application of International Application No. PCT/JP2019/016656 filed Apr. 18, 2019, the disclosure of which is incorporated herein by reference in its entirety. Further, this application claims priority from Japanese Patent Application No. 2018-080277, filed Apr. 18, 2018, the disclosure of which is incorporated herein by reference in their entirety.

### TECHNICAL FIELD

Technology disclosed herein relates to an image processing method, a program, and an image processing device.

### RELATED ART

Japanese Patent Application Laid-Open (JP-A) No. 2007-135868 discloses technology for generating a still image from a moving image imaged in fluorescent contrast imaging.

### SUMMARY

An image processing method of technology disclosed herein includes extracting a first frame from a first moving image of an examined eye and extracting a second frame from a second moving image of the examined eye, and comparing the first frame against the second frame.

A program of technology disclosed herein causes a computer to execute the image processing method of technology disclosed herein.

An image processing device of technology disclosed herein includes an image processing unit that executes extracting a first frame from a first moving image of an examined eye and extracting a second frame from a second moving image of the examined eye, and comparing the first frame against the second frame.

### BRIEF DESCRIPTION OF DRAWINGS

FIG. 1 is a block diagram of an ophthalmic system 100 of a first exemplary embodiment.

FIG. 2 is a schematic configuration diagram illustrating an overall configuration of an ophthalmic device 110.

FIG. 3 is a block diagram of a configuration of an electrical system of a management server 140.

FIG. 4 is a functional block diagram of a CPU 162 of a management server 140.

FIG. 5 is a sequence chart illustrating operations of an ophthalmic system 100 before and after treatment by a doctor.

FIG. 6 is a diagram illustrating a viewer screen 300 of an image viewer 150.

FIG. 7 is a diagram illustrating an analysis screen 300A of an image viewer 150.

FIG. 8 is a diagram illustrating a manner in which a distal end of a blood vessel present in a first selected image GA and in a second selected image GB is enlarged, and displayed by switching-display.

FIG. 9 is a diagram illustrating an analysis screen 300B of a second exemplary embodiment.

FIG. 10A is a diagram illustrating a distal end 402 (green) of a blood vessel 400 as a first color selected image IMA.

FIG. 10B is a diagram illustrating a distal end 404 (purple) of this blood vessel 400 as a second color selected image IMB.

FIG. 10C is a diagram illustrating an image synthesizing the distal end 402 (green) and the distal end 404 (purple).

FIG. 11 is a diagram illustrating an analysis screen 400A of an image viewer 150 of a first modified example.

FIG. 12 is a sequence chart for a second modified example and illustrates operations of an ophthalmic system 100 before and after treatment by a doctor.

### DETAILED DESCRIPTION

Detailed description follows regarding exemplary embodiment in the present invention, with reference to the drawings. Note that in the following, for ease of explanation, a scanning laser ophthalmoscope is referred to as an “SLO”.

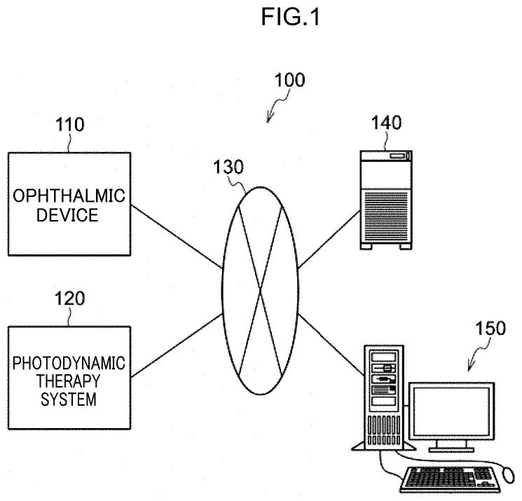

A configuration of the ophthalmic system 100 will now be described, with reference to FIG. 1. As illustrated in FIG. 1, the ophthalmic system 100 includes an ophthalmic device 110, an photodynamic therapy system 120, a management server device (hereafter referred to as a “management server”) 140, and an image display device (hereafter referred to as an “image viewer”) 150. The ophthalmic device 110 acquires fundus images. The photodynamic therapy system 120 performs photodynamic therapy (PDT) on an examined eye of a patient. The management server 140 stores plural fundus images and eye axial lengths obtained by imaging the fundi of plural patients using the ophthalmic device 110, by storing in association with respective patient IDs.

Icons and buttons are displayed on a display screen, described later, of the image viewer 150 for instructing the generation of images, also described later. When an ophthalmologist has clicked on an icon or the like, an instruction signal corresponding to the icon clicked is transmitted from the image viewer 150 to the management server 140. On receipt of the instruction signal from the image viewer 150, the management server 140 generates an image corresponding to the instruction signal and transmits image data of the generated image to the image viewer 150. The image viewer 150 that has received the image data from the management server 140 then displays an image based on the received image data on a display. Display screen generation processing is performed in the management server 140 by a display screen generation program to operate the CPU 162 being performed.

The management server 140 is an example of an “image processing device” technology disclosed herein.

The ophthalmic device 110, the photodynamic therapy system 120, the management server 140, and the image viewer 150 are connected to each other over a network 130.

Note that other ophthalmic instruments (examination instruments such as for field of view measurement, intraocular pressure measurement) and a diagnostic support device to perform image analysis using artificial intelligence may also be connected over the network 130 to the ophthalmic device 110, the photodynamic therapy system 120, the management server 140 and the image viewer 150.

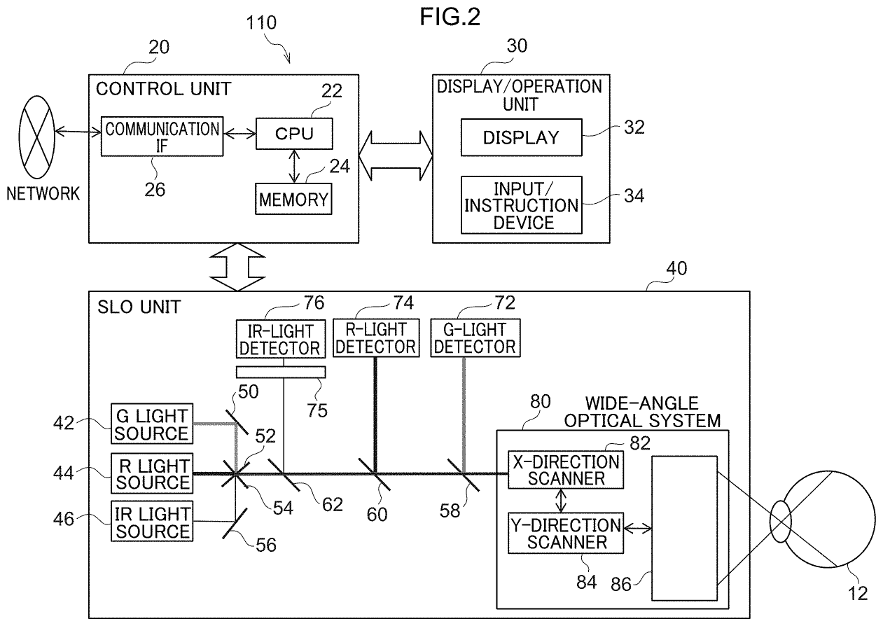

Next, description follows regarding a configuration of the ophthalmic device 110, with reference to FIG. 2. As illustrated in FIG. 2, the ophthalmic device 110 includes a control unit 20, a display/operation unit 30, and an SLO unit 40, and images the posterior segment (fundus) of the examined eye 12. Moreover, a non-illustrated OCT unit may also be provided to acquire OCT data of the fundus.

The control unit 20 includes a CPU 22, memory 24, a communication interface (I/F) 26, and the like. The display/operation unit 30 is a graphical user interface to display images obtained by imaging, and to receive various instructions including an imaging instruction. The display/operation unit 30 also includes a display 32 and an input/instruction device 34 such as a touch panel.

The SLO unit 40 includes a light source 42 for green light (G-light: wavelength 530 nm), a light source 44 for red light (R-light: wavelength 650 nm), and a light source 46 for infrared radiation (IR-light (near-infrared light): wavelength 800 nm). The light sources 42, 44, 46 respectively emit light as commanded by the control unit 20. The R-light light source emits visible light of wavelengths from 630 nm to 780 nm, and the IR-light light source employs a laser light source emitting near-infrared light having a wavelength of 780 nm or above.

The SLO unit 40 includes optical systems 50, 52, 54 and 56 that reflect or transmit light from the light sources 42, 44 and 46 and guide the light into a single optical path. The optical systems 50 and 56 are mirrors, and the optical systems 52 and 54 are beam splitters. The G-light is reflected by the optical systems 50 and 54, the R-light is transmitted through the optical systems 52 and 54, and the IR-light is reflected by the optical systems 52 and 56, such that all are guided into a single optical path.

The SLO unit 40 includes a wide-angle optical system 80 for scanning light from the light sources 42, 44, 46 in two-dimensions across the posterior segment (fundus) of the examined eye 12. The SLO unit 40 includes a beam splitter 58 that, from out of the light from the posterior segment (fundus) of the examined eye 12, reflects the G-light and transmits light other than the G-light. The SLO unit 40 includes a beam splitter 60 that, from out of the light transmitted through the beam splitter 58, reflects the R-light and transmits light other than the R-light. The SLO unit 40 includes a beam splitter 62 that, from out of the light that has passed through the beam splitter 60, reflects IR-light. The SLO unit 40 is provided with a G-light detection element 72 to detect the G-light reflected by the beam splitter 58, an R-light detection element 74 to detect the R-light reflected by the beam splitter 60, and an IR-light detection element 76 to detect IR-light reflected by the beam splitter 62.

An optical filter 75 is provided between the beam splitter 62 and the IR-light detection element 76, for example in the vicinity of a region where light is incident to the IR-light detection element 76, and the optical filter 75 has a face with a surface area that covers the entire region. The optical filter 75 is moved by a non-illustrated moving mechanism controlled by the CPU 22 between a position where the face of the optical filter 75 covers the entire region referred to above, and a position where the face of the optical filter 75 does not cover the entire region referred to above. The optical filter 75 is a filter blocking IR light (wavelength 780 nm) emitted from the IR-light source 46, and letting fluorescent light (wavelength 830 nm) emitted from ICG, described later, pass through.

The wide-angle optical system 80 includes an X-direction scanning device 82 configured by a polygon mirror to scan the light from the light sources 42, 44, 46 in an X direction, a Y-direction scanning device 84 configured by a galvanometer mirror to scan the light from the light sources 42, 44, 46 in a Y direction, and an optical system 86 including a non-illustrated slit mirror and elliptical mirror to widen the angle over which the light is scanned. The optical system 86 enables a field of view (FOV) of the fundus with a wider angle than in conventional technology to be achieved, enabling imaging of a fundus region over a wider range than when employing conventional technology. More specifically, the optical system 86 enables imaging of a fundus region over a wide range of approximately 120 degrees for an external light illumination angle from outside the examined eye 12 (in practice approximately 200 degrees about a center O of the eyeball of the examined eye 12 as a reference position for an internal light illumination angle where the fundus of the examined eye 12 can be imaged by illumination with scanning light). The optical system 86 may be configured employing plural lens sets instead of a slit mirror and elliptical mirror. The X-direction scanning device 82 and the Y-direction scanning device 84 may also each be a scanning device employing a two-dimensional scanner configured by MEMS mirrors.

A system using an elliptical mirror as described in International Applications PCT/JP2014/084619 or PCT/JP2014/084630 may be used in cases in which the optical system 86 is a system including a slit mirror and an elliptical mirror. The respective disclosures of International Application PCT/JP2014/084619 (International Publication WO2016/103484) filed on Dec. 26, 2014 and International Application PCT/JP2014/084630 (International Publication WO2016/103489) filed on Dec. 26, 2014 are incorporated by reference herein in their entireties.

Note that when the ophthalmic device 110 is installed on a horizontal plane, the “X direction” corresponds to a horizontal direction and the “Y direction” corresponds to a direction perpendicular to the horizontal plane. A direction connecting the center of the pupil of the anterior eye portion of the examined eye 12 and the center of the eyeball is referred to as the “Z direction”. The X direction, the Y direction, and the Z direction are accordingly perpendicular to one another.

The photodynamic therapy system 120 illustrated in FIG. 1 is a system to perform PDT by illuminating a weak laser beam onto a pathological lesion of a fundus after internal administration of a drug that reacts to light. PDT is performed to treat age-related macular degeneration and central serous retinopathy.

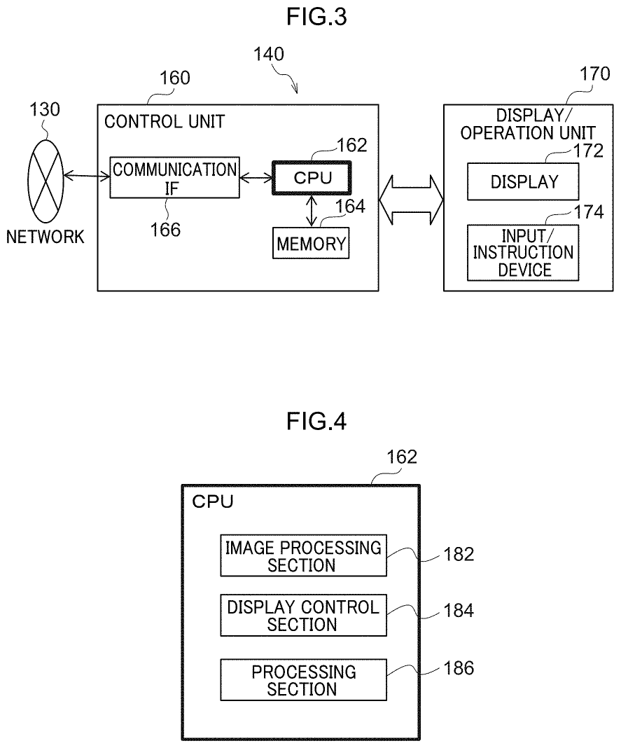

Next, a configuration of the management server 140 will be described with reference to FIG. 3. As illustrated in FIG. 3, the management server 140 includes a control unit 160, and a display/operation unit 170. The control unit 160 is equipped with a computer including a CPU 162, memory 164 configured by a storage device, a communication interface (I/F) 166, and the like. Note that an analysis processing program is stored in the memory 164. The display/operation unit 170 is a graphical user interface for displaying images and for receiving various instructions. The display/operation unit 170 includes a display 172 and an input/instruction device 174 such as a touch panel.

The configuration of the image viewer 150 is similar to that of the management server 140, and so description thereof is omitted.

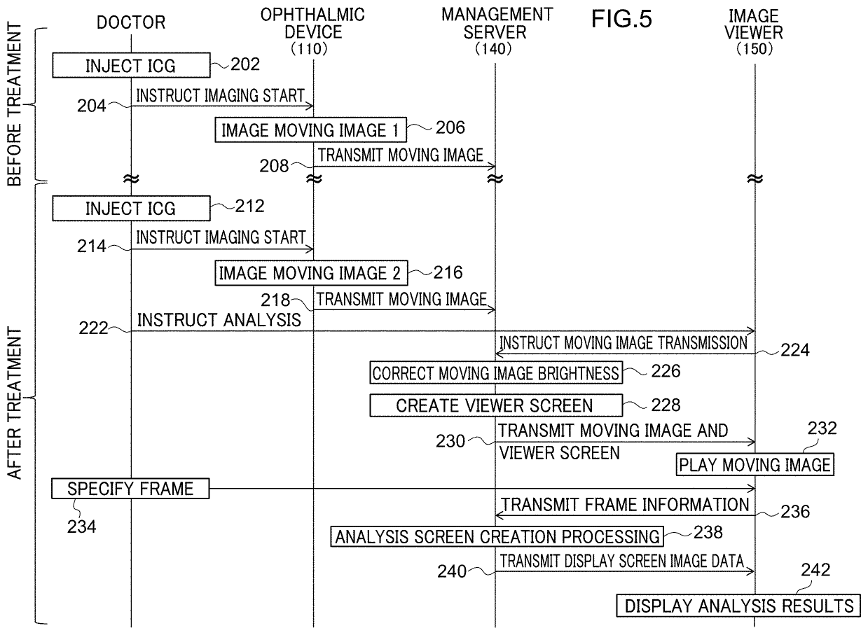

Next, with reference to FIG. 4, description follows regarding each of various functions implemented by the CPU 162 of the management server 140 executing the analysis processing program. The analysis processing program includes an analysis processing function, a display control function, and a processing function. By the CPU 162 executing the analysis processing program including each of these functions, the CPU 162 functions as an image processing unit 182, a display controller 184, and a processing unit 186, as illustrated in FIG. 4.

Next, with reference to FIG. 5, description follows regarding operation of the ophthalmic system 100 in which a doctor employs the photodynamic therapy system 120 to produce a visualization of a state of blood flow in choroidal blood vessels before and after treatment of, for example, age-related macular degeneration disease or central serous retinopathy.

Imaging of the examined eye of a patient is performed before treating the examined eye using PDT using the photodynamic therapy system 120. A specific example of this is described below.

The examined eye of the patient is positioned so as to allow imaging of the examined eye of the patient using the ophthalmic device 110. As illustrated in FIG. 5, at step 202, the doctor administers a fluorescent dye (contrast agent, indocyanine green (hereinafter, referred to as “ICG”)) into the body by intravenous injection. ICG is used to examine vascular lesions of the choroid since the excitation wavelength of ICG and the wavelength of fluorescent light therefrom are both in the near-infrared region. At step 204, the input/instruction device 34 is employed to instruct the ophthalmic device 110 to start fundus ICG fluoroscopy. At step 206 the ophthalmic device 110 starts fundus ICG fluoroscopy (imaging of a moving image 1) using the SLO unit 40. Specifically, the control unit 20 of the ophthalmic device 110 controls the IR light source 46 and the IR-light detection element 76 of the SLO unit 40 so as to image the fundus of the examined eye at a frequency of N (natural number) times per second over a specific period of time (T (natural number) seconds). Moving image data of the moving image 1 is accordingly generated by imaging the fundus of the examined eye in this manner for the specific period of time T at N frames per second. Thus N×T frames of images are obtained as the moving image 1. For example, N×T=10×60=600 frames of images are obtained.

When ICG has been intravenously injected, the ICG starts to flow through the blood vessels of the fundus after a fixed period of time has elapsed. When this occurs the ICG is excited by the IR-light (780 nm) from the IR light source 46, and the ICG emits fluorescent light having a wavelength (830 nm) in the near-infrared region. Moreover, when imaging the fundus of the examined eye (step 206) to generate the moving image data of moving image 1, the optical filter 75 (see FIG. 2) is inserted in front of and in the vicinity the region where light is incident onto the IR-light detection element 76. As described above, the optical filter 75 blocks the IR light (wavelength 780 nm) emitted by the IR light source 46, and lets the fluorescent light (wavelength 830 nm) emitted from ICG, described later, pass through. This means that only the fluorescent light emitted from the ICG is picked up by the IR-light detection element 76, enabling fundus ICG fluoroscopy (imaging of moving image 1) to be performed, and producing a visualization of the blood flow accompanying the ICG.

When imaging of moving image 1 (imaging of the examined eye for the specific period of time) has been completed, at the next step 208 the ophthalmic device 110 transmits the moving image (N×T frames) obtained by imaging moving image 1 to the management server 140.

After step 208, the photodynamic therapy system 120 subjects a specified site in the examined eye of the patient (pathological lesion) to PDT so as to treat the examined eye.

After treating the examined eye (for example, after 3 months have elapsed), imaging of the examined eye is performed again to confirm the efficacy of the treatment. This is specifically performed in the following manner.

The examined eye of the patient is positioned so as to enable imaging of the patient's examined eye using the ophthalmic device 110. At step 212, the ICG is administered internally by intravenous injection, and at step 214 the input/instruction device 34 is employed to instruct the ophthalmic device 110 to start imaging. At step 216, the ophthalmic device 110 performs imaging of a moving image 2. Note that since imaging moving image 2 at step 216 is similar to imaging moving image 1 at step 206, description thereof will be omitted. At step 218, the ophthalmic device 110 transmits the moving images (N×T frames) obtained by imaging the moving image 2 to the management server 140.

When imaging the moving image 1 at step 206 and when imaging the moving image 2 at step 216, various information is also input to the ophthalmic device 110, such as a patient ID, patient name, age, information as to whether each image is from the right or left eye, the date/time of imaging and visual acuity before treatment, and the date/time of imaging and visual acuity after treatment. The various information described above is transmitted from the ophthalmic device 110 to the management server 140 when the moving images are transmitted at step 208 and step 218.

At step 222, the user of the image viewer 150 (an ophthalmologist or the like) then uses the input/instruction device 174 of the image viewer 150 to instruct analysis processing to the management server 140 of the ophthalmic device 110.

At step 224, the image viewer 150 instructs the management server 140 to transmit moving images. At step 226, the management server 140 that has been instructed to transmit moving images reads moving image 1 and moving image 2 from the memory 164. The management server 140 then corrects the brightness value of each of the pixels of N×T frames of image in each of the moving image 1 and moving image 2 so as to eliminate the effect of fluctuations in background brightness.

More specifically, the effect of fluctuations in background brightness may be eliminated in the following manner. Namely, the image processing unit 182 may remove background brightness by computing an average brightness for each frame, and then dividing each of the pixel values of a given frame by the average brightness of that frame. The background brightness may also be removed by performing processing for each frame to divide signal values of each pixel by an average value of a region of fixed width surrounding that pixel. The image processing unit 182 then eliminates the effects of background brightness in a similar manner for all the other frames. Similar processing to moving image 1 is also executed on moving image 2.

At step 226, the image processing unit 182 may execute positional alignment to align positions of images in chronologically preceding and following frames of the N×T frames in each of moving image 1 and moving image 2. For example, the image processing unit 182 selects one specific frame from out of the N×T frames in either moving image 1 or moving image 2, for example moving image 1, as a reference frame. There are large fluctuations in the locations of contrasting blood vessels and signal strength in frames immediately after injection of ICG, and so a frame after a certain passage of time has elapsed and subsequent to sufficient ICG starting to perfuse through the arteries and veins is preferably selected as the reference frame.

The image processing unit 182 performs positional alignment using a method such as cross-correlation processing using the brightness values of the pixels in the frames so as to align feature points of the fundus region of a specific frame with these feature points on the fundus of the reference frame. The image processing unit 182 also, for the positions of all of the other frames, performs similar positional alignment of all of the other frames to the reference frame.

The image processing unit 182 also executes similar processing for the moving image 2 to the above positional alignment for moving image 1. Note that the image processing unit 182 may also be configured so as to correct positional misalignment of frames in moving image 1 and moving image 2 so as to align the position of feature points in the fundus image for all the frames of moving image 1 and moving image 2.

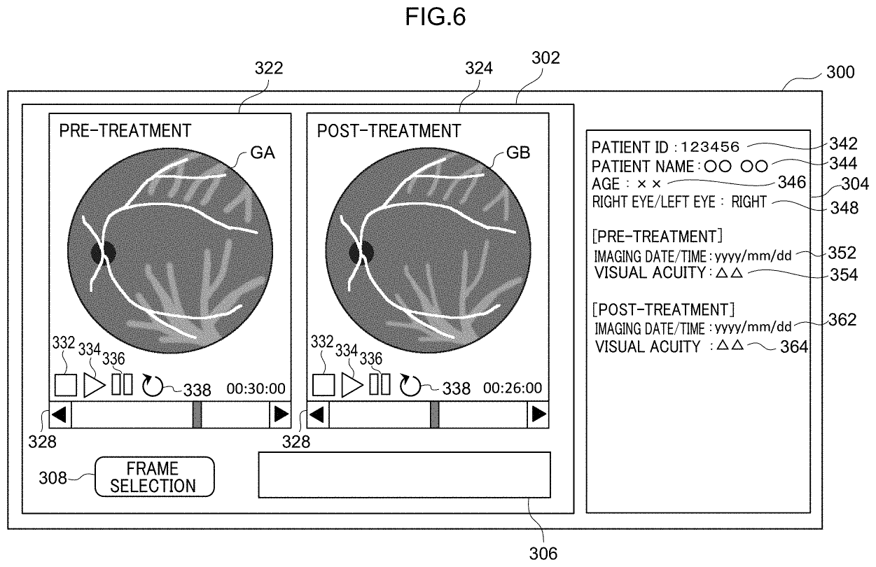

At step 228, the image processing unit 182 displays a viewer screen. FIG. 6 illustrates a viewer screen 300. As illustrated in FIG. 6, the viewer screen 300 includes an image display region 302 and a patient information display region 304.

A pre-treatment image display region 322 to display images from before treatment (the moving image 1), a post-treatment image display region 324 to display images from after treatment (the moving image 2), an information display region 306, and a frame selection icon 308 are provided in the image display region 302.

A stop icon 332 to instruct stopping of image (moving image 1) playback, a play icon 334 to instruct image playback, a pause icon 336 to instruct pausing of image playback, and a repeat icon 338 to instruct repeat of image playback are provided in the pre-treatment image display region 322. A current position display region 328 is provided in the pre-treatment image display region 322 to indicate that the image currently being displayed is an image of what position chronologically from out of moving image 1 overall. Note that an elapsed time (00:30:00) is displayed at a position adjacent to the current position display region 328 to indicate that the image currently being displayed is an image of however many seconds subsequent to the start of moving image 1. Note that, in sequence from a stop icon 332, a repeat icon 338, a current position display region 328, and an elapsed time (00:26:00) are also displayed in the post-treatment image display region 324.

A patient ID display region 342, a patient name display region 344, an age display region 346, a display region 348 to display information (left or right) to indicate whether each image is from the left eye or the right eye, a pre-treatment imaging date/time display region 352, a pre-treatment visual acuity display region 354, a post-treatment imaging date/time display region 362, and a post-treatment visual acuity display region 364 are provided in the patient information display region 304.

At step 230, the management server 140 transmits the respective data for the moving image 1 and moving image 2 and the viewer screen 300 to the image viewer 150. At step 232 the image viewer 150 displays the viewer screen 300 on the display 172.

At step 234, the doctor operates the viewer screen 300 to select frames for comparison. Selection of the frames for comparison is specifically performed in the following manner.

An image of a specific frame of the pre-treatment moving image 1, for example, the image of the final frame therein, is displayed in the image display region 302 of the viewer screen 300 (see FIG. 6). An image of a specific frame of the post-treatment moving image 2, for example, the image of the final frame therein, is displayed in the pre-treatment image display region 322. For example, the moving image 1 is played when the play icon 334 of the pre-treatment image display region 322 is operated. If the pause icon 336 is operated during playback of the moving image 1 then playback of the moving image 1 is stopped, and if the play icon 334 is then operated, playback of the moving image 1 is resumed from the stopped location. If the stop icon 332 is operated during playback of the moving image 1 then playback of the moving image 1 is stopped, and if the play icon 334 is then operated, the moving image 1 is played from the start. When the repeat icon 338 is operated, playback of the moving image 1 is repeated from the start. Playback of the moving image 2 is similar to playback of the moving image 1.

The user (ophthalmologist or the like) operates the play icon, the pause icon 336, and the repeat icon 338 to find the timing at which fluorescence is emitted in all of the blood vessels of the fundus being displayed in the pre-treatment image display region 322. The user (ophthalmologist or the like) then presses the pause button 336 at a timing when an image is being displayed in which all of the blood vessels of the fundus are emitting fluorescence, and stops playback of the moving image 1. The elapsed time and the frame number at the point in time when the pause icon 336 was pressed is temporarily saved in the memory 164 of the image viewer 150 as first frame information of the pre-treatment moving image. There are large fluctuations in the locations of contrasting blood vessels and signal strength in frames immediately after injection of ICG, and so a frame after a certain passage of time has elapsed and subsequent to sufficient ICG starting to perfuse through the arteries and veins is selected. Then at step 232, when the user (ophthalmologist or the like) presses the frame selection icon 308, first frame information that is being temporarily stored is transmitted to the management server 140.

Selection of a frame in the post-treatment image display region 324 is performed similarly to the selection of a frame in the pre-treatment image display region 322, and is transmitted as post-treatment moving image second frame information to the management server 140 (steps 234, 236).

FIG. 6 illustrates an example in which the user (ophthalmologist or the like) has selected a first frame at t=30 seconds elapsed of the pre-treatment moving image 1, and has selected a second frame at t=26 seconds elapsed of the post-treatment moving image 2. The images of the frames from before treatment and after treatment are respectively selected in this manner by the user (ophthalmologist or the like).

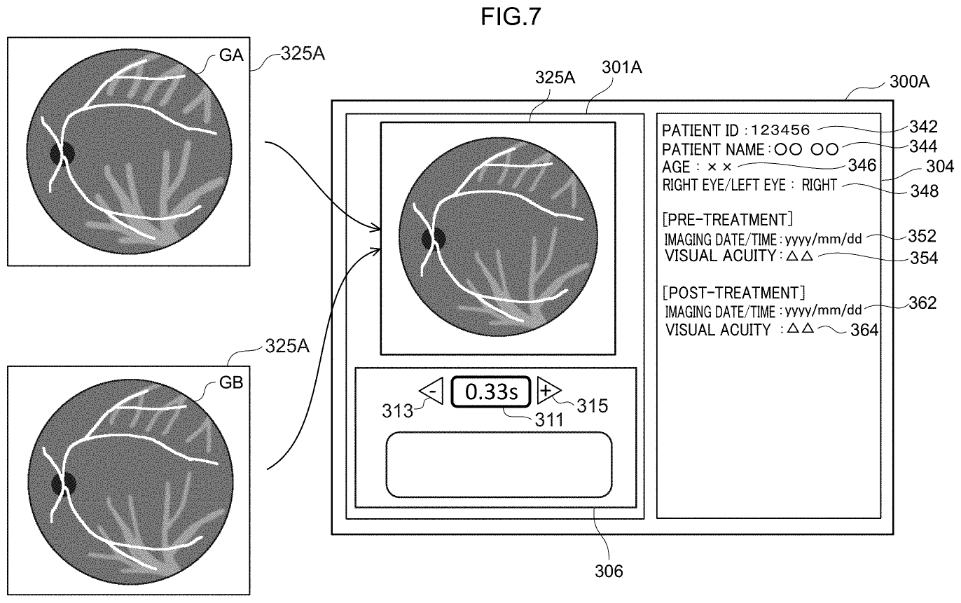

At step 238, the image processing unit 182 of the management server 140 creates the analysis screen 300A (see FIG. 7). As illustrated in FIG. 7, the analysis screen 300A includes an information display region 301A, and a patient information display region 304 similar to the patient information display region 304 of the viewer screen 300 in FIG. 6.

The information display region 301A includes a selected image switching-display region 325A to display the selected image, and an information display region 306. The information display region 306 includes an interval display section 311 to display a switching interval time for switching between a first selected image GA and a second selected image GB, and a plus icon 315 and a minus icon 313 to adjust the switching interval time so as to be longer or shorter, respectively.

The image processing unit 182 extracts from the first frame information transmitted at step 232 a first frame corresponding to the first frame information in the moving image 1, and extracts from the second frame information a second frame corresponding to the second frame information in the moving image 2. The image processing unit 182 creates the first selected image GA based on the extracted first frame, and creates the second selected image GB based on the extracted second frame.

In the present exemplary embodiment, the first selected image GA generated based on the first frame and the second selected image GB generated based on the second frame are displayed on the selected image switching-display region 325A by switching-display while alternately switching therebetween at the specific interval.

In the present exemplary embodiment, the image processing unit 182 executes positional alignment on the first selected image GA and the second selected image GB prior to switching-display at the specific interval. The eye position is not fixed between frames within the moving images due to differences in the imaging position before treatment and after treatment, and due to eye movements during moving image imaging. The effects of eye movements are eliminated by performing positional alignment of the first selected image GA and the second selected image GB when switching-display is performed.

As method for positional alignment, for example, corresponding points may be detected between images by template matching or the like using cross correlation based on brightness values, and then the position of one of the images positionally aligned based on the positions of the detected corresponding points so as to align the corresponding points. By positionally aligning the first selected image GA and the second selected image GB, the fundus images are not displayed with misalignment as the images are switched in the selected image switching-display region 325A. Namely, the locations where there are changes between the first selected image GA and the second selected image GB are made apparent to the user (ophthalmologist or the like) by performing the switching-display.

At step 240, the management server 140 transmits to the image viewer 150 the display screen image data for the analysis screen 300A (see FIG. 7) including the positionally aligned first selected image GA and second selected image GB. Note that in order to display the first selected image GA and the second selected image GB alternately, an animation GIF file using the graphics interchange format (GIF) may be embedded in the analysis screen 300A, so as to perform switching-display on the image viewer 150.

At step 242, the image viewer 150 displays analysis results. Specifically the image viewer 150 displays the analysis screen 300A on the display 172. More specifically, the image viewer 150 displays the first selected image GA and the second selected image GB in the selected image switching-display region 325A of the analysis screen 300A by switching therebetween at the specific interval.

The switching interval time of FIG. 7 may be freely settable by the user (ophthalmologist or the like) by selectively pressing the plus icon 315 and the minus icon 313 to adjust the switching interval time.

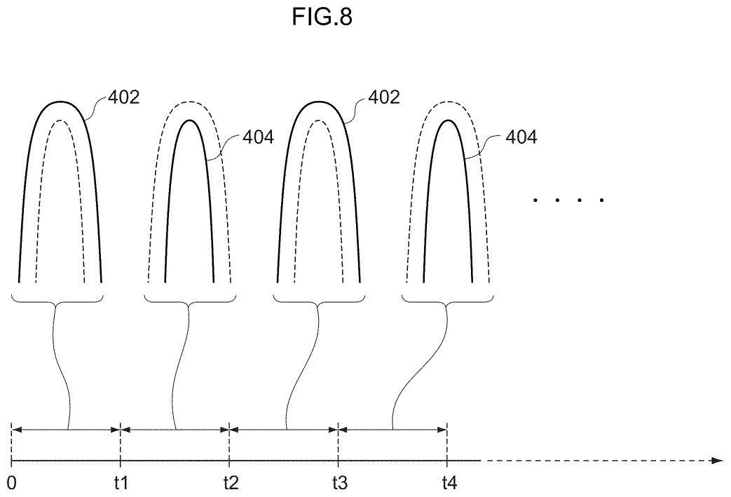

FIG. 8 illustrates a manner in which a distal end of a blood vessel respectively present in the first selected image GA and the second selected image GB is enlarged and switching-displayed. As illustrated in FIG. 8, a pre-treatment blood vessel 402 in the first selected image GA is displayed in the selected image switching-display region 325A from a time 0 to a time t1 (interval T), and a post-treatment blood vessel 404 in the second selected image GB is displayed therein from time t1 to time t2 (interval T). The pre-treatment blood vessel 402 in the first selected image GA is displayed therein from time t2 to time t3 (interval T), and the post-treatment blood vessel 404 in the second selected image GB is displayed therein from time t3 to time t4 (interval T). Similarly from then onwards the first selected image GA (the pre-treatment blood vessel 402) and the second selected image GB (the post-treatment blood vessel 404) are switching-displayed in the selected image switching-display region 325A by alternating at each interval T.

If the treatment is effective, then the thickness of the blood vessel differs between before treatment and after treatment. Thus the doctor looking at the pre-treatment blood vessel 402 and the post-treatment blood vessel 404 of different thicknesses as they are switching-displayed in the selected image switching-display region 325A is able to interpret this as the treatment being effective.

Hitherto, a still image is generated from a moving image imaged by fluorescent contrast imaging. It has not been able produce a visualization using a fundus image of the efficacy of treatment using conventional technology.

Moreover, in the present exemplary embodiment, the first selected image GA and the second selected image GB are switching-displayed, and so if the thickness is different between the pre-treatment blood vessel 402 and the post-treatment blood vessel 404 then this appears as an increase in size and a decrease in size in the switching-display. The doctor looking at this is able to interpret the efficacy of treatment. The present exemplary embodiment accordingly enables visualization using a fundus image of the efficacy of treatment.

Next, description follows regarding a second exemplary embodiment. The configuration of the second exemplary embodiment is similar to the configuration of the first exemplary embodiment, and so description thereof will be omitted. The operation of the second exemplary embodiment is substantially similar to the operation of the first exemplary embodiment, and so only different operation will be explained. The operation of the second exemplary embodiment differs from the operation of the first exemplary embodiment in the content from step 238 to step 242 of FIG. 5.

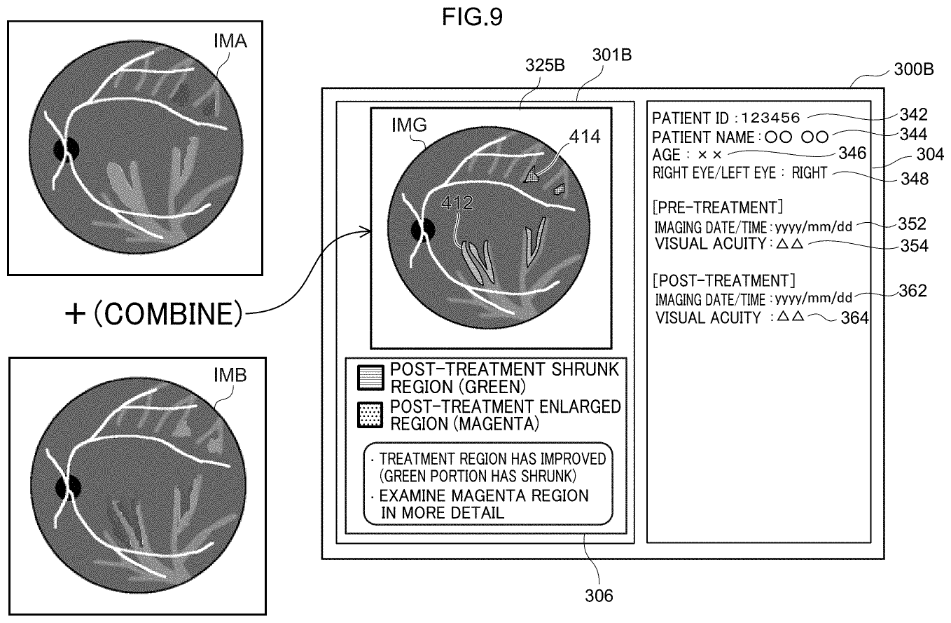

At step 238 in FIG. 5 of the first exemplary embodiment, the image processing unit 182 of the management server 140 creates the analysis screen 300A (see FIG. 7). However, in the second exemplary embodiment, the image processing unit 182 creates the analysis screen 300B (see FIG. 9). As illustrated in FIG. 9, the analysis screen 300B includes an information display region 301B and a patient information display region 304 as described above.

The information display region 301B includes a synthesized image display region 325B to display a synthesized image from synthesizing a pre-treatment image and a post-treatment image, and an information display region 306.

In the present exemplary embodiment, similarly to in the first exemplary embodiment, the image processing unit 182 performs positional alignment on the first selected image GA and the second selected image GB.

In the present exemplary embodiment, the image processing unit 182 colors the positionally-aligned first selected image GA and second selected image GB. More specifically, the image processing unit 182 converts the image data of the first selected image GA that is only brightness data into RGB data with 0 for both an R component and a B component, and brightness values the same as the original brightness data for a G component. The image processing unit 182 converts the image data of the second selected image GB that is only brightness data into RGB data with brightness data the same as the original brightness data for an R component and a B component, and with 0 as the G component. The monochrome first selected image GA is thereby converted into a green first color selected image IMA, and the monochrome second selected image GB is thereby converted into a purple (magenta) second color selected image IMB.

As described later, a synthesized image IMG synthesizing the first color selected image IMA and the second color selected image IMB is displayed in the synthesized image display region 325B.

The image processing unit 182 creates, as messages to display on the information display region 306, messages of content to attract attention. The messages “region which shrunk after treatment (green)”, “region which enlarged after treatment (magenta)”, “treatment region has improved (green portion has shrunk)”, and “examine the magenta region in more detail” are created.

At step 240 in the present exemplary embodiment, the management server 140 transmits to the image viewer 150 image data of a display screen for the analysis screen 300B (see FIG. 9) that includes the synthesized image IMG synthesizing the positionally-aligned and colored first color selected image IMA and second color selected image IMB.

At step 242 of the present exemplary embodiment, the image viewer 150 displays the analysis results. Specifically, the image viewer 150 displays the analysis screen 300B on the display 172. More specifically, the image viewer 150 displays the synthesized image IMG in the synthesized image display region 325B of the analysis screen 300B.

The image viewer 150 displays in the information display region 306 the messages to attract attention of “region which shrunk after treatment (green)”, “region which enlarged after treatment (magenta)”, “treatment region has improved (green portion has shrunk)”, and “examine the magenta region in more detail”.

The user (ophthalmologist or the like) accordingly examines the green portions 412 or the purple regions 414 of the synthesized image IMG displayed on the synthesized image display region 325B.

The user (ophthalmologist or the like) also examines the magenta portions 414 in the synthesized image display region 325B. The magenta portions 414 are enlarged regions where blood vessels not present before treatment are enlarged, or where the blood vessels have been enlarged by treatment. Thus the presence of magenta portions 414 enables the user (ophthalmologist or the like) to interpret these as portions of where blood vessels are enlarged compared to before treatment.

In the second exemplary embodiment, an image synthesizing the first color selected image IMA and the second color selected image IMB is displayed. Thus when the pre-treatment blood vessels and the post-treatment blood vessels have different thicknesses, portions that are now thinner than before treatment are displayed in color (green) and portions that are thicker than before treatment are displayed in color (magenta). The user (ophthalmologist or the like) looking at this is thereby able to interpret the efficacy of treatment. The present exemplary embodiment enables a visualization using a fundus image to be produced in this manner of the efficacy of treatment.

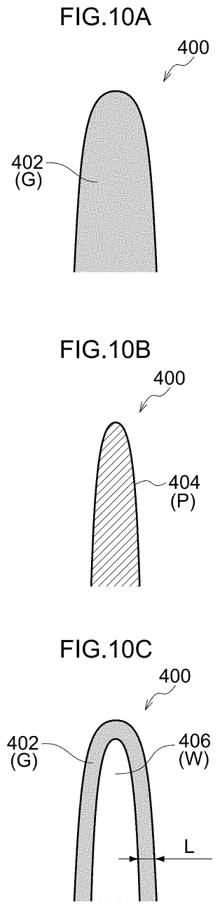

FIG. 10A illustrates the distal end 402G (green) of the blood vessel 400 that is the first color selected image IMA, and FIG. 10B illustrates the distal end 404 (purple) of the blood vessel 400 that is the second color selected image IMB. FIG. 10C illustrates an image synthesizing the distal end 402G (green) of the blood vessel 400 that is the first color selected image IMA with the distal end 404P (purple) of the blood vessel 400 that is the second color selected image IMB. As illustrated in FIG. 10C, an overlap portion 406W where the distal end 402 (green) and the distal end 404 (purple) overlap is white, and a non-overlap portion where the distal end 402 (green) and the distal end 404 (purple) do not overlap remains green. Namely, the RGB data of the pixels of the distal end 402G of FIG. 10A is only G data, and the RGB data of the pixels of the distal end 404P of FIG. 10B is only R and B data. Synthesizing such respective pixel data of the only G RGB data of the 402G pixels with the RGB data of the pixels of the 406W in the synthesized image of FIG. 10C results in a display in white (greyscale of only brightness information) due all of the R, G, and B components being present.

As described above, when the treatment is effective, then for the thickness of the blood vessels in the fundus as illustrated in FIG. 10C, the thickness of the post-treatment blood vessel 404 is thinner by a length L than the thickness of the pre-treatment blood vessel 402. Thus when an image has a green image region around a white image region then this can be interpreted as the treatment being effective. Thus the doctor looking at the superimposed image in an superimposed image display region 322R is able to interpret the treatment as being effective when there is a green image region around a white image region.

Next, description follows regarding various modified examples of the technology disclosed herein.

### First Modified Example

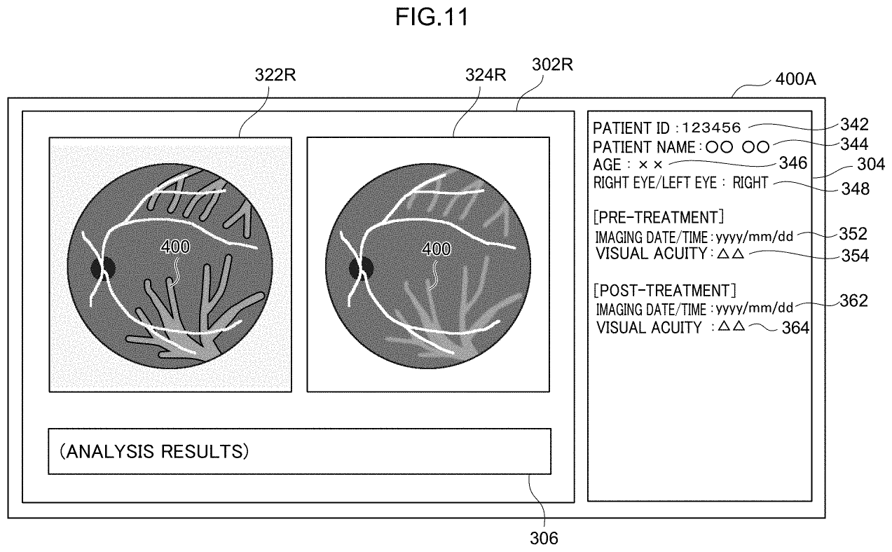

At step 242 of FIG. 5 in the first exemplary embodiment, the first selected image GA and the second selected image GB are switching-displayed, and at step 242 of FIG. 5 in the second exemplary embodiment, an image synthesizing the first color selected image IMA and the second color selected image IMB is displayed. However, the technology disclosed herein is not limited thereto. For example, as illustrated in FIG. 11, a synthesized image display region 322R and switching-display region 324R may be provided in an image display region 302R on an analysis screen 400A. An image synthesizing the first color selected image IMA and the second color selected image IMB is displayed in the synthesized image display region 322R. The first selected image GA and the second selected image GB are switching-displayed in the switching-display region 324R.

Thus at step 238 of FIG. 5 of the first modified example, both the processing of step 238 for the first exemplary embodiment and the processing of step 238 for the second exemplary embodiment are executed.

### Second Modified Example

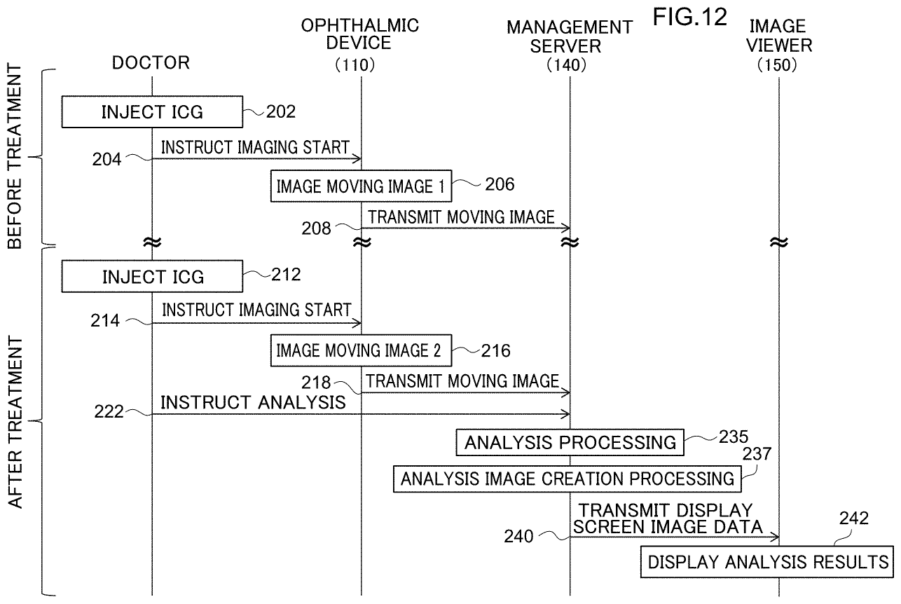

In the first exemplary embodiment and the second exemplary embodiment, the doctor selects the frames to be compared from out of the N×T frames in moving image 1 and moving image 2 (see step 234 of FIG. 5). However, technology disclosed herein is not limited thereto, and, for example, instead of a doctor selecting frames to compare, the selection may be made automatically by an artificial intelligence (AI) embedded in the management server 140. FIG. 12 illustrates the operation of the ophthalmic system 100 by a doctor before and after treatment in cases in which the frames for comparison are selected automatically by an AI. The same reference numerals are appended to the operation of FIG. 12 as the reference numerals for the same operation in FIG. 5, and description thereof is omitted.

As illustrated in FIG. 12, after the doctor has instructed analysis at step 222, the respective processing from step 224 to step 236 is omitted, and at step 235 the management server 140 executes analysis processing to select the frames for comparison automatically using the AI. For example, the AI may be configured to ascertain a trend, in moving image 1 and moving image 2, to a gradually larger number of pixels changing in the direction of brightness increases as the ICG starts to flow into a blood vessel, followed by an absence of pixels of changing brightness, before an increase in the number of pixels that change in the direction of brightness decrease. The AI selects a frame in the period when there is an absence of pixels of changing brightness. At step 237, the analysis screen 300A of FIG. 7 or the analysis screen 300B (see FIG. 9) or the analysis screen 400A of FIG. 11 are created, and with the images of the reference frames being the images of frames selected by the AI.

### Third Modified Example

In the first exemplary embodiment, the first selected image GA and the second selected image GB are acquired, and in the second exemplary embodiment the first color selected image IMA and the second color selected image IMB are created, however, the following analysis processing may also be executed.

As described above, when the treatment is effective, then as illustrated in FIG. 10C, for the thickness of a blood vessel of the fundus, the thickness of the post-treatment blood vessel 404 is thinner by a length L than the thickness of the pre-treatment blood vessel 402.

In this analysis processing, the image processing unit 182 extracts each of the blood vessel from the images of the respective frames, calculates the thickness of each of the extracted blood vessels therein, calculates the difference between the thickness of the respective blood vessels before and after treatment, and then determines whether or not the calculated difference is greater than a predetermined threshold. The image processing unit 182 stores the treatment in the memory 164 as being effective for cases in which the calculated difference exceeded the threshold, and stores the treatment in the memory 164 as not being effective for cases in which the calculated difference did not exceed the threshold. The management server 140 then transmits the data regarding the efficacy or otherwise of the treatment to the image viewer 150. Based on the received data, the image viewer 150 displays whether or not the treatment was effective.

Moreover, there is no limitation to displaying whether or not the treatment was effective and, for example, instead of displaying whether or not the treatment was effective, “examination not required” or “examination required” may be displayed. Or a configuration may be adopted in which “continued treatment not required” or “continued treatment required” is displayed instead of “examination not required” or “examination required. Or a configuration may be adopted in which “number of treatment sessions” is displayed instead of “examination required” or “continued treatment required”. Note that the “number of treatment sessions” may be calculated from the thickness of the blood vessel post-treatment and the difference in the thickness of blood vessel before and after treatment (i.e. the reduction in thickness corresponding to one session of treatment) and the target thickness of blood vessels.

### Fourth Modified Example

In the first exemplary embodiment and the second exemplary embodiment the moving image 1 and moving image 2 are acquired before and after one session of treatment, however the technology disclosed herein is not limited thereto. For example, before and after moving images may be acquired for each of plural sessions of treatment, and the analysis processing executed using the before and after moving images for each treatment session so as to display the results thereof.

### Fifth Modified Example

In the first exemplary embodiment, switching is performed between the first selected image GA and the second selected image GB, however the technology disclosed herein is not limited thereto, and switching-display may be performed using the first color selected image IMA and the second color selected image IMB.

In each of the examples above, the first selected image GA is employed for the green first color selected image IMA and the second selected image GB is employed for the purple second color selected image IMB, however the technology disclosed herein is not limited thereto. For example, the first selected image GA may be employed for the purple first color selected image IMA, and the second selected image GB may be employed for the green second color selected image IMB.

Moreover, although green and purple have a complementary color relationship to each other, the complementary color relationship is not limited to green and purple, and colors of another complementary color relationship may be employed. Note that pairs of colors not having a complementary color relationship to each other may also be employed.

### Sixth Modified Example

In the exemplary embodiments described above examples have been described in which a fundus image is acquired by the ophthalmic device 110 with an internal light illumination angle of about 200 degrees. However, the technology disclosed herein is not limited thereto, and the technology disclosed herein may, for example, be applied even when the fundus image imaged by an ophthalmic device has an internal illumination angle of 100 degrees or less.

### Seventh Modified Example

In the exemplary embodiments described above the ophthalmic device 110 uses SLO to image an ICG moving image. However, the technology disclosed herein is not limited thereto, and for example an ICG moving image imaged with a fundus camera may be employed.

### Eighth Modified Example

The exemplary embodiment described above describes an example of the ophthalmic system 100 equipped with the ophthalmic device 110, the photodynamic therapy system 120, the management server 140, and the image viewer 150; however the technology disclosed herein is not limited thereto. For example, as a first example, the photodynamic therapy system 120 may be omitted, and the ophthalmic device 110 may be configured so as to further include the functionality of the photodynamic therapy system 120. Moreover, as a second example, the ophthalmic device 110 may be configured so as to further include the functionality of one or both of the management server 140 and the image viewer 150. For example, the management server 140 may be omitted in cases in which the ophthalmic device 110 includes the functionality of the management server 140. In such cases, the analysis processing program is executed by either the ophthalmic device 110 or the image viewer 150. Moreover, the image viewer 150 may be omitted in cases in which the ophthalmic device 110 includes the functionality of the image viewer 150. As a third example, the management server 140 may be omitted, and the image viewer 150 may be configured so as to execute the functionality of the management server 140.

### Ninth Modified Example

Although in the exemplary embodiments described above photodynamic therapy (PDT) is employed as the treatment, the technology disclosed herein is not limited thereto. The technology disclosed herein may be employed to confirm an effect between before and after treatment for various pathological changes related to the fundus, such as treatment by photocoagulation surgery, treatment by administration of anti-VEGF drugs, treatment by surgery on the vitreous body, and the like.

### Other Modified Examples

The data processing described in the exemplary embodiments described above are merely examples thereof. Obviously, unnecessary steps may be omitted, new steps may be added, and the sequence of processing may be changed within a range not departing from the spirit thereof.

Moreover, although in the exemplary embodiments described above examples have been given of cases in which data processing is implemented by a software configuration utilizing a computer, the technology disclosed herein is not limited thereto. For example, instead of a software configuration utilizing a computer, the data processing may be executed solely by a hardware configuration of FPGAs or ASICs. Alternatively, a portion of processing in the data processing may be executed by a software configuration, and the remaining processing may be executed by a hardware configuration.

1.-10. (canceled)

11. An image processing method comprising:
acquiring a first frame extracted from a first moving image that images a fundus of an examined eye;
acquiring a second frame extracted from a second moving image that images a fundus of the examined eye in a timing different from a timing of the first moving image; and
comparing a first blood vessel displayed in the first frame and the second blood vessel displayed in the second frame.

12. The image processing method of claim 11, wherein comparing include comparison of a size of the first blood vessel and a size of the second blood vessel.

13. The image processing method of claim 11, further comprising performing positional alignment of the first frame and the second frame.

14. The image processing method of claim 13, wherein the performing positional alignment includes detecting corresponding points between the first frame and the second frame by template matching using cross correlation based on brightness values and aligning the detected corresponding points.

15. The image processing method of claim 11, further comprising:
generating a synthesized image synthesizing the first frame and the second frame; and
outputting the synthesized image.

16. The image processing method of claim 15, wherein the generating the synthesized image includes:
creating a first color image from the first frame;
creating a second color image from the second frame wherein the second color is different from the first color; and
synthesizing the first color image and the second color image.

17. The image processing method of claim 11, wherein the displaying includes displaying the first frame and the second frame alternately at a specific time interval.

18. The image processing method of claim 11, wherein the displaying includes displaying the blood vessel in the first frame and the second frame in enlarged form.

19. The image processing method of claim 18, wherein the displaying includes displaying a distal end of the blood vessel in the first frame and the second frame in enlarged form.

20. The image processing method of claim 11, wherein the displaying includes:
displaying a portion that the thickness of the blood vessel imaged in the second frame is thinner than the thickness of the blood vessel imaged in the first frame in first color; and
displaying a portion that the thickness of the blood vessel imaged in the second frame is thicker than the thickness of the blood vessel imaged in the first frame in second color.

21. The image processing method of claim 11, wherein the first moving image is a moving image imaging a fundus before treatment and the second moving image is a moving image imaging the fundus after treatment.

22. The image processing method of claim 21, wherein the displaying includes displaying efficacy of the treatment based on a difference between a thickness of the blood vessel in the first frame and a thickness of the blood vessel in the second frame.

23. The image processing method of claim 21, wherein the displaying includes displaying a necessity for examination, a necessity for continued treatment, and/or a number of treatment sessions based on a difference between a thickness of the blood vessel in the first frame and a thickness of the blood vessel in the second frame.

24. The image processing method of claim 21, wherein the first moving image and the second moving image are moving images imaging the fundus with fluoroscopy.

25. The image processing method of claim 11, wherein the first frame is a frame in the first moving image in which the entire first blood vessel is fluorescing, and the second frame is a frame in the second moving image in which the entire second blood vessel is fluorescing.

26. The image processing method of claim 11, further comprising:
processing the first moving image by performing positional alignment between chronologically preceding and following frames of a plurality of frames obtained from the first moving image at a first timing; and
processing the second moving image by performing positional alignment between chronologically preceding and following frames of a plurality of frames obtained from the second moving image at a second timing.

27. The image processing method of claim 11, wherein the acquiring the first frame and the second frame includes:
displaying the first moving image, the second moving image, a first moving image stop icon, and a second moving image stop icon on a screen; and
receiving first frame information of a time at which the first moving image stop icon is pressed and second frame information of a time at which the second moving image stop icon is pressed.

28. The image processing method according to claim 11, wherein the first moving image and the second moving image are moving images that visualize the blood flow state of the choroidal blood vessels of the examined eye.

29. A non-transitory computer-readable storage medium storing a program to cause a computer to execute the image processing method comprising:
acquiring a first frame extracted from a first moving image that images a fundus of an examined eye;
acquiring a second frame extracted from a second moving image that images a fundus of the examined eye in a timing different from a timing of the first moving image; and
comparing a first blood vessel displayed in the first frame and a second blood vessel displayed in the second frame.

30. An image processing device comprising:
a memory;
a processor connected to the memory and that:
acquire a first frame extracted from a first moving image that images a fundus of an examined eye;
acquire a second frame extracted from a second moving image that images a fundus of the examined eye in a timing different from a timing of the first moving image; and
compare a first blood vessel displayed in the first frame and a second blood vessel displayed in the second frame.
